(19) United States
(12) Patent Application Publication
(10) Pub. No.: US 2009/0148946 A1
(43) Pub. Date: Jun. 11, 2009

(54) SEPARATION SYSTEM FOR CELL, AND CELL CULTURE SYSTEM USING SEPARATOR FOR CELL, AND METHOD OF SEPARATION FOR CELL

(76) Inventors: Ryoichi HAGA, Hitachi (JP); Keisuke Shibuya, Tokai (JP); Masaru Namba, Hitachinaka (JP)

Correspondence Address: BRUNDIDGE & STANGER, P.C., 1700 DIAGONAL ROAD, SUITE 330, ALEXANDRIA, VA, 22314, US

(21) Appl. No.: 12/330,743

(22) Filed: Dec. 9, 2008

(30) Foreign Application Priority Data
Dec. 10, 2007 (JP) 2007-318597

(51) Int. Cl. C12N 5/06; C12M 1/36

(52) U.S. Cl. 435/379; 435/286.5

(57) ABSTRACT

A cell is separated from a culture broth with an extremely simple configuration, and production yield of a target product is improved. There are provided separating means for separating a cell from the culture broth containing the cell, a buffer tank connected to the separating means through a first pipeline, at least one or more liquid reservoir tank connected to the buffer tank through a second pipeline, a first valve disposed on the first pipeline and controlling communication between the separating means and the buffer tank, and a second valve disposed on the second pipeline and controlling communication between the buffer tank and the liquid reservoir tank.

### BACKGROUND OF THE INVENTION

1. Field of the Invention

The present invention relates to a separation system for cell for separating a cell from a culture broth of a living cell, and a cell culture system using a separator for cell, and a method of separation for cell for separating a cell from a cell culture broth.

2. Background Art

In culture of a living cell, it is desired that a living cell is cultured with a high density in order to improve productivity of a target product, and a continuous culture method is one of culture methods to achieve this object. In continuous culture of an animal cell, an operation to separate a cell in a culture broth and extract only a liquid component from a culture vessel and to supply a new medium is indispensable, and various cell separating means have been developed so far.

“Tissue Culture”, pp. 283 to 287, No. 8, Volume 15 (issued in 1989) and JP Patent Publication (Kokai) No. 6-209761 A (1994) describe a continuous culture device applying settling using gravity. JP Patent Publication (Kokai) No. 5-192607 A (1993) and JP Patent Publication (Kokai) No. 6-90737 A (1994) describe a method of settling a cell using centrifugal separation. JP Patent Publication (Kokai) No. 5-95778 A (1993) describes a culture device in which a culture region surrounded by a filter material preventing passage of a cell is provided in a culture vessel and a filtrate having passed through is pulled out to outside the culture region. JP Patent Publication (Kokai) No. 1-281072 A (1989) describes use of a porous hollow fiber membrane as a filter membrane and washing the membrane by back stream using a medium. JP Patent Publication (Kokai) No. 6-98758 A (1994) describes installation of two pairs of filter membranes, one of which is used as ventilating means and the other as filtering means, and repetition of filtering and washing by back stream by changing them alternatively. JP Patent Publication (Kohyo) No. 03-505041 A (1991) describes a culture device in which a rotating filter membrane is provided in a culture vessel. JP Patent Publication (Kokai) No. 06-237754 A (1994) describes a culture device in which a rotating filter membrane is installed outside a culture vessel.

However, these prior-art methods have respective serious problems. That is, with the method of settling using gravity, a settling speed of a cell is small and a settling zone should be made larger, whose application to a large-sized culture device is extremely difficult. The centrifugal separating device has complicated device configuration and its size increase is difficult. The filter separating device has simple device configuration and its scale-up is easy, but clogging of the filter membrane is substantially unavoidable, and a separation operation for a long time is difficult.

Moreover, when the prior-art method is to be put into practice in an actual plant scale, it has a problem that there is no usable pump or flow regulating valve. In order to be applied to a culture plant, the system is required to have a high-level sealing property in order to prevent intrusion of microorganisms, to withstand a washing operation using a washing fluid containing strong alkali or strong acid as its main component and moreover, to be constituted by a structure and a material capable of complete sterilization inside by having steam at 130° C., 0.2 MPa flown. Pumps satisfying the above conditions include a tube roller pump. However, the tube roller pump is used in desk-top scale devices in many cases, but there is a fear of breakage in a tube, and it is difficult to be applied to a plant for pharmaceutical manufacture and the like requiring high-level reliability. Also, most of the valves used in the culture plant are operated in two stages of fully-open and fully-closed such as a diaphragm valve, and there is substantially no valve functioning as a flow regulating valve within a small flow-rate range.

### SUMMARY OF THE INVENTION

Then, in view of the above circumstances, the present invention has an object to provide a separation system for cell that can separate a cell from a culture broth with extremely simple configuration and improve production yield of a target product, a culture system provided with the separation system for cell, and a method of separation for cell.

The present invention that has achieved the above-mentioned objects includes the following:

That is, the separation system for cell according to the present invention comprises separating means for separating the cell from a culture broth containing the cell, a buffer tank connected to the separating means through a first pipeline, at least one or more liquid reservoir tanks connected to the buffer tank through a second pipeline, a first valve disposed on the first pipeline for controlling communication between the separating means and the buffer tank, and a second valve disposed on the second pipeline for controlling communication between the buffer tank and the liquid reservoir tank. In the separation system for cell according to the present invention, the first pipeline is released by the first valve to have the separating means and the buffer tank communicate with each other, the culture broth is aspirated to the separating means by an internal pressure difference between the buffer tank and the separating means, and the first pipeline is blocked by the first valve, while a second pipeline is released by the second valve to have the buffer tank and the liquid reservoir tank communicate with each other, and the culture broth after cell has been separated can be transferred to the liquid reservoir tank by the internal pressure difference between the buffer tank and the liquid reservoir tank.

Also, the separation system for cell according to the present invention preferably further comprises a liquid reservoir tank for washing by back stream connected to the buffer tank through a third pipeline and a third valve disposed on the third pipeline and controlling communication between the buffer tank and the liquid reservoir tank for washing by back stream. In this case, in the separation system for cell according to the present invention, the third pipeline is released by the third valve so as to have the buffer tank and the liquid reservoir tank for washing by back stream communicate with each other, a solution for washing by back stream is supplied to the buffer tank by an internal pressure difference between the buffer tank and the liquid reservoir tank for washing by back stream, the third pipeline is blocked by the third valve and the first flow passage is released by the first valve so as to have the buffer tank and the separating means communicate with each other, and the solution for washing by back stream in the buffer tank is supplied to the separating means by the internal pressure difference between the buffer tank and the separating means.

Moreover, as the separating means in the separation system for cell according to the present invention, such means containing a rotating filter having a filter for blocking passage of a cell on an outer circumferential face of a cylindrical rotor in a vessel provided with a culture-broth inflow port and a culture-broth discharge port can be used.

Furthermore, the separating means in the separation system for cell according to the present invention is preferably arranged in the culture vessel into which a medium for culturing a cell is injected.

On the other hand, the cell culture system using separator for cell according to the present invention comprises the above-mentioned separation system for cell according to the present invention and a culture vessel in which a culture broth of a living cell is filled. In the cell culture system using separator for cell according to the present invention, the separating means in the above-mentioned separation system for cell can separate a filtrate not containing the cell from the culture broth containing the cell and filled in the culture vessel.

Also, the method of separation for cell according to the present invention, using the separation system for cell comprising: separating means for separating a cell from the culture broth containing the cell; a buffer tank connected to the separating means through a first pipeline; at least one or more liquid reservoir tank connected to the buffer tank through a second pipeline; a first valve disposed on the first pipeline for controlling communication between the separating means; and the buffer tank, and a second valve disposed on the second pipeline and controlling communication between the buffer tank and the liquid reservoir tank, releases the first pipeline by the first valve so as to have the separating means and the buffer tank communicate with each other, aspirates the culture broth to the separating means by an internal pressure difference between the buffer tank and the separating means, blocks the first pipeline by the first valve and releases the second pipeline by the second valve so as to have the buffer tank and the liquid reservoir tank communicate with each other so as to transfer the culture broth after the cell is separated by the internal pressure difference between the buffer tank and the liquid reservoir tank to the liquid reservoir tank.

Moreover, in the method of separation for cell according to the present invention, it is preferable that using the separation system for cell further comprising a liquid reservoir tank for washing by back stream connected to the buffer tank through a third pipeline and a third valve disposed on the third pipeline for controlling communication between the buffer tank and the liquid reservoir tank for washing by back stream, the third pipeline is released by the third valve so as to have the buffer tank and the liquid reservoir tank for washing by back stream communicate with each other, a solution for washing by back stream is supplied to the buffer tank by an internal pressure difference between the buffer tank and the liquid reservoir tank for washing by back stream, the third pipeline is blocked by the third valve and the first flow passage is released by the first valve so as to have the buffer tank and the separating means communicate with each other and to supply the solution for washing by back stream in the buffer tank to the separating means by the internal pressure difference between the buffer tank and the separating means.

Moreover, in the method of separation for cell according to the present invention, the separating means in the separation system for cell containing a rotating filter having a filter for blocking passage of a cell on an outer circumferential face of a cylindrical rotor in a vessel provided with the inflow port and the discharge port is preferably used.

Moreover, in the method of separation for cell according to the present invention, the separating means in the separation system for cell is preferably disposed in the culture vessel.

According to the separation system for cell, the cell culture system using separator for cell, and the method of separation for cell according to the present invention, a cell can be separated from a culture broth with extremely simple configuration. As a result, efficient culture is achieved, and production yield of the target product can be improved.

### BRIEF DESCRIPTION OF THE DRAWINGS

FIG. 1 is an outline block diagram schematically illustrating an example of a separation system for cell to which the present invention is applied.

FIG. 2 is an outline block diagram schematically illustrating an example of a cell culture system using a separator for cell provided with the separation system for cell to which the present invention is applied.

### DESCRIPTION OF SYMBOLS

1 filtration filter container
2 culture broth
3 filtration filter
4 filter
5 buffer tank
6a, 6b filtrate transfer pipeline
7 filtrate tank
8 medium tank
9 medium transfer pipeline

### DETAILED DESCRIPTION OF THE PREFERRED EMBODIMENTS

A separation system for living cell, a method of separation for cell, and a cell culture system using separator for cell according to the present invention will be described below in detail referring to the attached drawings. The separation system for living cell, the method of separation for cell, and the cell culture system using separator for cell according to the present invention can be applied to culture of cells producing substances to be a main material of pharmaceuticals and the like. In the present invention, the substances to be produced are not limited at all but include proteins such as antibody, enzyme and the like and physiologically active substances such as lowmolecule substances, macromolecule substances and the like. Cells to be cultured are not limited at all but include animal cell, plant cell, insect cell, bacteria, yeast, fungi, algae and the like. Particularly, the separation system for living cell, the method of separation for cell, and the cell culture system using separator for cell according to the present invention are preferably intended for culture of animal cells for producing proteins such as antibody, enzyme and the like.

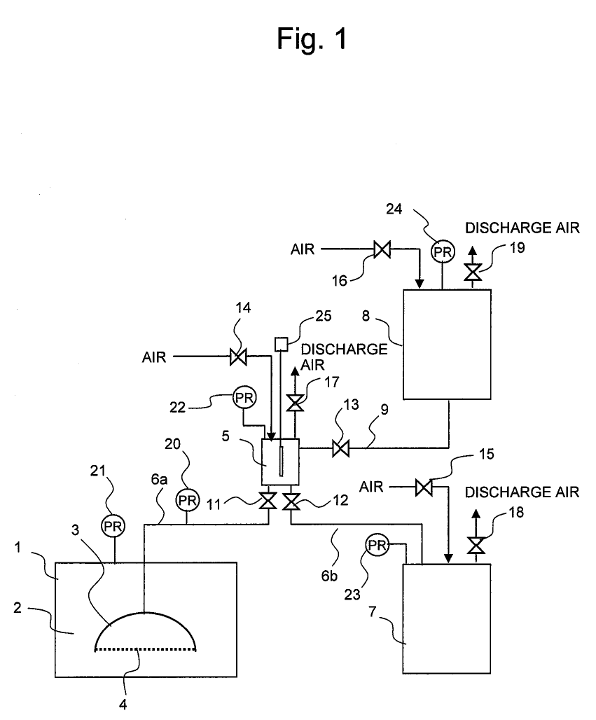

An example of the separation system for cell to which the present invention is applied is schematically shown in FIG. 1. The separation system for cell to which the present invention is applied comprises a filtration filter 3 provided with a filter 4 for blocking passage of a cell on one end face in contact with culture broth, a filtration filter container 1 for containing the filtration filter 3, a buffer tank 5 connected to the filtration filter 3, a filtrate tank 7 connected to the buffer tank 5, and a medium tank 8 connected to the buffer tank 5. The filtration filter 3 and the buffer tank 1 are made to communicate through a filtrate transfer pipeline 6a (first pipeline), the buffer tank 5 and the filtrate tank 7 are made to communicate through a filtrate transfer pipeline 6b (second pipeline), and the buffer tank 5 and the medium tank 8 are made to communicate through a medium transfer pipeline 9 (third pipeline), respectively. At each of the transfer pipelines, a valve 11 (first valve), a valve 12 (second valve), and a valve 13 (third valve) are provided, respectively. Here, the valves 11, 12, and 13 which are operated in two stages of fully open or fully closed are preferably used.

The filtrate transfer pipeline 6a having the filtration filter 3 and the buffer tank 5 communicate with each other comprises a barometer 20 for detecting a pressure in the pipeline. The filtration filter container 1 is provided with a barometer 21 for detecting a pressure inside. The buffer tank 5 is provided with a barometer 22 for detecting a pressure inside. The filtrate tank 7 is provided with a barometer 23 for detecting a pressure inside. The medium tank 8 is provided with a barometer 24 for detecting a pressure inside.

Moreover, in the buffer tank 5, a valve 14 for introducing air from outside and a valve 17 for adjusting pressure inside (also referred to as a pressure regulating valve 17 in some cases) are disposed. In the buffer tank 5, a liquid level meter 25 for measuring a liquid level such as a medium filled inside is disposed. In the filtrate tank 7, a valve 15 for introducing air from outside and a valve 18 for adjusting a pressure inside (also referred to as a pressure regulating valve 18 in some cases) are disposed. In the medium tank 8, a valve 16 for introducing air from outside and a valve 19 for adjusting a pressure inside (also referred to as a pressure regulating valve 19 in some cases) are disposed. For the air supplied by these valves 14 to 19, aseptic air from which micro organisms such as bacteria are removed in advance is preferably used.

The above-mentioned separation system has configuration in which the culture broth 2 is supplied into the filtration filter container 1 by communicating means, not shown. Thus, the pressure inside the filtration filter container 1 measured by the barometer 21 is the same as that of a culture vessel. According to the communicating means, not shown, the culture broth 2 inside the filtration filter container 1 can circulate between it and the culture vessel. As a result, even if filtrate is extracted by filtration, a bad influence on the culture caused by deterioration in the environment due to partial rise in density of the cell and accumulation for a long time can be prevented. The separation system for cell according to the present invention is not limited to configuration having the communicating means, not shown, but may be configured so that the filtration filter container 1 or the filtration filter 3 is immersed in a medium filled in the culture vessel. If the filtration filter container 1 or the filtration filter 3 is immersed in the medium, the above-mentioned deterioration in the environment due to partial rise in the cell density or accumulation for a long time can be prevented by agitation of the medium in the culture vessel. If the filtration filter 3 is immersed in the medium filled in the culture vessel, the filtration filter container 1 is synonymous with the culture vessel.

By using the separation system for cell configured as above, the cell contained in the medium can be separated, and the medium after separation of the cell can be recovered. A filtering process, a filtrate discharge process, a medium transfer process, and a washing by back stream process using the separation system for cell will be described below in this order.

### (1) Filtering Process

The filtering process is a process of separating a cell from a medium during culture (or after culture has been finished) and of recovering a medium not containing the cell in the buffer tank 5. First, culture broth is circulated in the filtration filter container 1. A pressure of the filtration filter container 1 is measured by the barometer 21, and the pressure inside the buffer tank 5 is measured by the barometer 22. The pressure of the filtration filter container is the same as the pressure of the culture vessel, and it is usually pressurized at 0.01 to 0.05 MPa. The pressure inside the buffer tank 5 at start of the filtering process is set at the same as the pressure of the filtration filter container. Usually, the internal pressures of the both tanks are made the same by an operation to press-feed air into the filtration filter 3 from the buffer tank 5 in the washing by back stream process, which will be described later. If the internal pressures of the both tanks are different, they are made the same by air injection by opening of the valve 14 and adjustment of the pressure regulating valve 17.

Though the filtrate tank 7 is usually pressurized to 0.01 to 0.05 MPa by air injection by opening of the valve 15 and adjustment of the pressure regulating valve 18, it is adjusted to be an internal pressure lower than the pressure of the culture vessel. In carrying out the filtering process, first, the valve 11, the valve 12, the valve 13, the valve 14, and the valve 17 provided at the buffer tank 5 are closed. Then, the valve 12 provided at the filtrate transfer pipeline 6b is opened for a short time and closed immediately after that, and the liquid, air or a mixture of them in the buffer tank 5 is discharged to the filtrate tank 7. By this operation, the internal pressure of the buffer tank 5 is slightly lowered to a pressure lower than the internal pressures of the filtration filter container 1 and the culture vessel. Next, the valve 11 provided in the filtrate transfer pipeline 6a is opened for a short time and closed immediately after that, and the liquid, air or a mixture of them inside the filtrate transfer pipeline 6a is discharged to the buffer tank 5. By this operation, the pressure inside the filtrate transfer pipeline 6a is slightly lowered and as a result, the pressure inside the filtration filter 3 communicating with the filtrate transfer pipeline 6a becomes slightly lower than the pressure of the filtration filter container 1, and the culture broth flows into the filtration filter 3 through fine pores of the filter 4. At this time, since a living cell larger than the fine pore of the filter 4 is prevented from passing, the cells are removed from the liquid flowing into the filtration filter 3, that is, the filtrate. That is, by repeating the opening/closing operation of the valve 11 and the valve 12, the filtrate obtained by removing the cells from the culture broth can be obtained.

The shape of the buffer tank 5 is not particularly limited but it may be spherical or cylindrical. Also, a pipe diameter of the pipeline between the valve 11 and the valve 12 may be expanded to have the buffer tank 5. A mounting method and a mounting position of the filtrate transfer pipeline 6a and the filtrate transfer pipeline 6b to the buffer tank 5 are not limited but they are preferably arranged at a bottom section of the tank so that there is no remaining liquid when the liquid in the buffer tank 5 is discharged. Also, the filtrate transfer pipeline 6a and the filtrate transfer pipeline 6b are preferably connected to the buffer tank 5, respectively, but such structure may be employed that the both are bonded and the middle of the valve 11 and the valve 12 is made to communicate with the buffer tank 5 via a single pipeline. A distance between the buffer tank 5 and the valve 11 as well as the valve 12 is preferably as small as possible. Particularly, the distance between the valve 12 and the buffer tank 5 is preferably given consideration to be minimized. A capacity of the buffer tank 5 is determined on the basis of a size of the cell culture system to be applied.

Opening/closing of the valve 11 and the valve 12 is preferably alternated, and attention should be paid so that the both valves are not opened at the same time in the filtering process. If the both valves are opened at the same time a pressure fluctuation buffering action of the buffer tank 5 can not be obtained, but a pressure difference between the filtration filter container 1 and the filtrate tank 7 is directly applied on the filter 4 and causes leakage of the living cell into the filtrate and clogging of the fine pores.

Adjustment of a filtration speed is made by adjusting an opening/closing frequency of the valve 11 and the valve 12, and an open time of each valve is basically made constant in operation. Increase of the valve open time inevitably increases pressure fluctuation and promotes clogging, which is not preferable.

### (2) Filtrate Discharge Process

The filtrate discharge process is a process of transferring the filtrate after removal of the cells recovered in the buffer tank 5 to the filtrate tank 7. In this process, first, the valve 14 is opened so as to inject air into the buffer tank 5 and the pressure inside the buffer tank 5 is raised higher than the internal pressure of the filtrate tank 7. When the internal pressure of the buffer tank 5 reaches a predetermined value, the culture broth recovered into the buffer tank 5 by opening the valve 12 can be transferred to the filtrate tank 7 through the filtrate transfer pipeline 6b. The open time of the valve 14 and the valve 12 in this process is determined by capacity of the buffer tank 5 and specification of the filtrate transfer pipeline 6b, and it is preferably determined usually by conducting a liquid passing test in advance.

### (3) Medium Transfer Process

The medium transfer process is a process of supplying the medium filled in the medium tank 8 to the buffer tank 5. In this process, first, the internal pressure of the buffer tank 5 is adjusted by the pressure regulating valve 17 lower than the internal pressure of the medium tank 8. The internal pressure of the medium tank 8 is usually pressurized to 0.01 to 0.05 MPa. Next, by opening the valve 13, the medium in the medium tank 8 flows into the buffer tank 5. A liquid level position of the medium is measured by the liquid level meter 25 provided at the buffer tank 5, and when it reaches a predetermined position, the valve 13 is closed, and supply of the medium is finished. In setting the pressure, consideration should be given to the liquid level heights of the medium tank 8 and the buffer tank 5. That is, if the liquid level position of the medium tank 8 is higher than the liquid level position of the buffer tank 5, a pressure difference is preferably set small so that a transfer speed of the medium is not increased. If the liquid level position of the medium tank 8 is lower than the liquid level position of the buffer tank 5, the pressure difference is preferably set large so that the medium is transferred. If the transfer speed of the medium needs to be adjusted, the adjustment can be made by carrying out opening/closing of the valve 13 intermittently so as to adjust a length of the open time.

### (4) Washing by Back Stream Process

The washing by back stream process is a process of washing the filter 4 by flowing the culture broth, a liquid for washing by back stream, air or a mixture of them in a direction opposite to a flowing direction of the medium in the above filtering process with respect to the filter 4. Specifically, the washing by back stream process can be carried out by supplying the medium injected into the buffer tank 5 in the above medium transfer process to the filtration filter 3. In this process, first, the valve 14 is opened and air is injected into the buffer tank 5, and the internal pressure of the buffer tank 5 is set higher than the internal pressure of the filtration filter container 1. Next, by opening the valve 11, the medium injected into the buffer tank 5 is injected into the filtration filter 3 through the filtrate transfer pipeline 6a and passes the fine pores of the filter 4 in a direction opposite to that in the filtering process. The medium having passed the filter 4 flows into the filtration filter container 1 and is supplied into the medium tank. That is, when the medium is supplied to the medium tank, the medium plays a role as a washing liquid using back stream of the filter 4.

In the washing by back stream process, by keeping on opening the valve 11, all the mediums in the buffer tank 5 are pushed out by a pressure of air and all the mediums in an internal space of the buffer tank 5, the filtrate transfer pipeline 6a, and the filtration filter 3 can be discharged into the filtration filter container 1. That is, without wasting expensive mediums, all of them can be made available for culture. After all the mediums are discharged, passage of the air used for pressurization through the fine pores of the filter 4 carries out washing by back stream of the filter by air. The air blown into the filtration filter container 1 is supplied into the culture vessel through the communication pipeline with the culture vessel. The supply of a large quantity of air into the culture vessel violently stirs the liquid level, with which a foam layer on an upper face of the culture broth is also stirred, and an action to promote break of foam is obtained.

After the washing by back stream by air supply is carried out for a predetermined time, the valve 14 is closed and the washing by back stream process is finished. At this time, by taking time from the closing operation of the valve 14 to the closing operation of the valve 11, the internal pressures of the filtration filter container 1 and the buffer tank 5 can be made the same. The time difference in the closing operation between the valve 14 and the valve 11 largely depends on properties of the filter 4 pore size, area, material and the like, and it is determined by conducting a test in advance.

By repeating the operations (1) to (4) described above, the separation operation of the living cell can be continued. Particularly, according to the separation system for cell according to the present invention, without requiring complicated configuration such as a pump device, the above (1) filtering process and the (2) filtrate discharge process as well as the above (3) medium transfer process and the (4) washing by back stream process can be carried out with extremely simple system configuration.

In this separation system for cell, the filter 4 is not particularly limited as long as it can block passage of a cell such as generally-used filter cloth, membrane filter and the like. Particularly, those having mechanical strength, heat resistance and corrosion resistance that can withstand injection of a washing liquid at sterilization or washing by blowing a steam into the filtration filter 3 are preferably selected. Also, since a large quantity of fine cell debris generated due to collapse of dead cells are present in the culture broth, a filter having a filtering characteristic to block passage of sound living cells and to pass fine cell debris is particularly preferable.

The liquid level meter 25 is not particularly limited but those having mechanical strength, heat resistance and corrosion resistance that can withstand injection of a washing liquid at sterilization or washing by blowing a steam are preferably selected. Also, since a large quantity of fine cell debris generated due to collapse of dead cells are present in the culture broth and there is a fear that foams are accumulated on the liquid level, a meter without a fear of malfunction caused by stain on a sensor is preferably selected. From such point of view, an electrostatic capacity type level sensor is preferably used as the level meter 25.

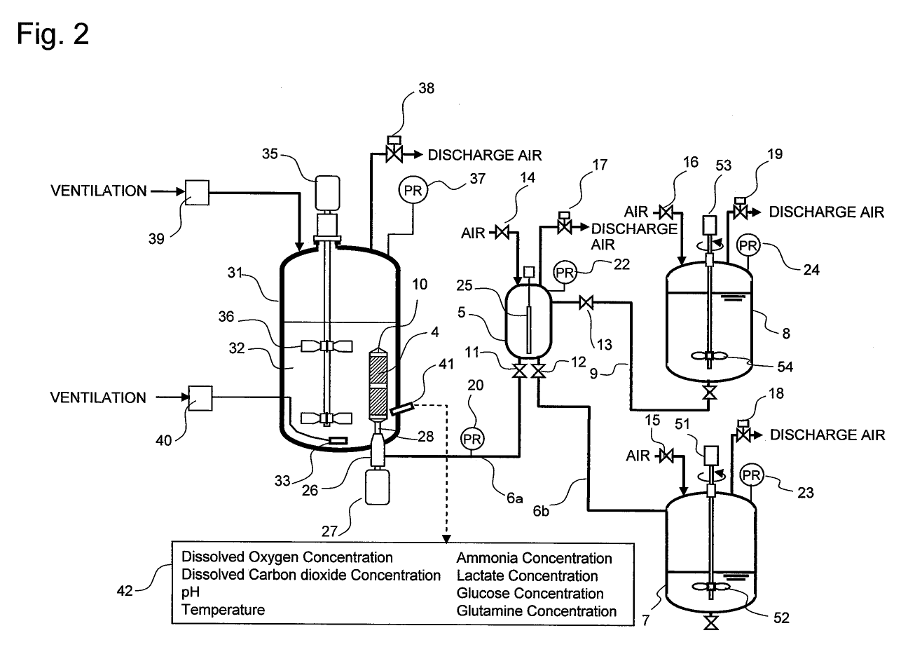

Also, the separation system for cell according to the present invention can be applied to a cell culture system using a separator for cell provided with a culture vessel in which a culture broth of a living cell is filled. That is, an outline configuration diagram of the cell culture system using a separator for cell according to the present invention is shown in FIG. 2. In the cell culture system using a separator for cell shown in FIG. 2, the same reference numerals are given to the same members and the same configuration as those in the separation system for cell shown in FIG. 1 and the detailed description will be omitted.

The cell culture system using a separator for cell shown in FIG. 2 is provided with a culture vessel 31, the buffer tank 5, the medium tank 8, and the filtrate tank 7. In the culture vessel 31, the filtration filter 3 is incorporated. The filtration filter 3 and the buffer tank 5 are made to communicate with each other by the filtrate transfer pipeline 6a, the buffer tank 5 and the filtrate tank 7 through the filtrate transfer pipeline 6b, and the buffer tank 5 and the medium tank 8 through the medium transfer pipeline 9, respectively. At each of the transfer pipelines, the valve 11, the valve 12, and the valve 13 are provided.

Though not shown in FIG. 2, gas supply equipment such as air, oxygen, nitrogen, carbon dioxide gas and the like, hot water/cold water supply equipment, steam supply equipment, and water supply/discharge equipment are provided.

The culture vessel 31 is shown in a section. The culture broth 32 filled in the culture vessel 31 is stirred by a stirrer 36 driven by a motor 35 for driving and mixed uniformly. Oxygen required for culture is supplied by two methods of a gas babbling aeration for supplying oxygen-containing gas into the liquid from sparging means 33 arranged on the bottom section of the tank and a surface aeration for ventilating to a gas-phase section on the upper part of the tank.

The culture vessel 31 is provided with measuring means 41 for measuring properties of the culture broth 32, and measured values 42 of dissolved oxygen concentration, dissolved carbon dioxide gas concentration, pH, temperature, ammonia concentration, lactate concentration, and glutamine concentration are obtained. As for the measuring means 41, one detecting means is used for each detection item or control item in an actual system, but only one of them is described in FIG. 2 for simplification.

Also, the culture vessel 31 is provided with individual operating means 40 and 39 for controlling supply gas in respective ventilation systems into the liquid and to the upper face. The individual operating means 39 and 40 are provided with a flow-rate control function and a supply-amount measuring function for each gas of air, oxygen and carbon dioxide, respectively. As for the ventilation to the upper face, its composition and a ventilation amount are controlled by the individual operating means 39 for controlling the supply gas. In this embodiment, air is ventilated in a predetermined amount and a carbon dioxide gas is mixed according to pH of the culture broth. In the control of the carbon dioxide gas concentration, using pH as a control amount, usual proportional control is executed with the carbon dioxide flow-rate as an operation factor. In the ventilation from the sparging means 37 into the culture broth, its composition and a ventilation amount are controlled by the individual operating means 40 for controlling the supply gas. In this embodiment, the dissolved oxygen concentration in the culture broth is used as a control amount and the oxygen ventilation amount is used as the operation factor.

The culture vessel 31 is maintained at a predetermined pressure by the pressure regulating valve 38 on the basis of the measurement result of the barometer 37. Usually, it is pressurized to 0.01 to 0.05 MPa in order to prevent intrusion of bacteria and the like from outside.

At the culture vessel 31, filtration filtering means for separating a cell from the culture broth is provided. The filtration filtering means in this embodiment is not particularly limited but a rotating filtration filter 10 provided with a filter 4 for blocking passage of a cell on the outer circumferential face of a cylindrical rotor coaxial with a rotating shaft 28 rotated by a driving motor 27 can be used. The rotating filtration filter 10 has a structure in which the cylindrical filter 4 is mounted on the rotating shaft 28, and an upper end and a lower end are sealed by a sealing member, filtering is carried out by making the pressure inside lower than outside, and filtrate having passed the fine pores of the filter 4 is obtained. As the filter 4, a metal filter with a slit-like opening section formed by winding a stainless thin wire cylindrically with a predetermined interval can be used. In such metal filter, fine pores other than the above slit do not substantially exist. As a result, the filter can block passage of the cells and pass fine particles such as cell debris smaller than the cell. A slit width is determined by a size of the cell and it is usually 5 to 30 μm. In use of this filter, a filtering differential pressure is preferably managed appropriately. That is because even if the slit width is larger than a diameter of the cell, if a large filtering differential pressure is applied, the cell might be deformed and pass through the slit or cause clogging.

In the filtering process, when the rotating filtration filter 10 is rotated in the culture vessel 31, a flow of the culture broth in parallel with the surface of the filter 4 is generated, and so-called cross-flow filtration state is brought about. By having a turbulent flow state on the surface of the filter 4, a higher-density layer of cells and fine particles formed by extraction of the filtrate can be diffused. As a result, clogging of the filter can be suppressed, and a larger quantity of the filtrate can be obtained at a higher speed. A rotation speed of the rotating filtration filter 10 is determined by resistance of the living cell to be cultured against a physical external force and a shape of the separation system for cell. It is usually operated at 100 to 1000 rpm.

In order to rotate the rotating filtration filter 10, a function to prevent outflow of the culture broth and intrusion of bacteria from outside is preferably provided. Thus, at the culture vessel 31, a shaft seal 26 using a mechanical seal is provided. The mechanical seal blocks leakage of a gas or a liquid by sliding a slide seal fixed to the rotating shaft 28 and rotating and a slide seal fixed to the shaft seal 26 in close contact with each other. In this cell culture system using a separator for cell, two mechanical seals of a mechanical seal for preventing outflow of the culture broth in the rotating filtration filter 10 and a mechanical seal for preventing intrusion from outside are used, and the filtrate transfer pipeline 6a is connected between the two mechanical seals. Also, the rotating shaft 28 has a hollow structure with one end opened at a position opposed to the filtrate transfer pipeline 6a and the other end opened inside the rotating filtration filter 10 so that the structure becomes a flow passage for connecting inside of the rotating filtration filter 10 and the filtrate transfer pipeline 6a to each other. In this cell culture system using a separator for cell, the mechanical seal is not particularly limited but any seal that can maintain air-tightness such as a mechanical seal for supplying seal water used in a usual cell culture system using a separator for cell, a dry-type mechanical seal and the like can be used. Also, a usage, arrangement and the like of the mechanical seal are not particularly limited.

The filtrate tank 7 is a tank for storing a filtrate in which a substance with a production purpose separated in the rotating filtration filter 10 is dissolved and may be provided with a stirrer 52 and a motor 51 for driving stirrer. Also, the filtrate tank 7 is preferably provided with a cooling device that can cool a target substance at 5 to 10° C. in order to prevent alteration of the target substance during storage. The medium tank 8 is a tank for storing a medium to be supplied to the culture vessel 31 and may be provided with a stirrer 54 and a motor 53 for driving stirrer. Also, the medium tank 8 is preferably provided with a cooling device that can cool the medium at 5 to 10° C. in order to prevent alteration of the medium during storage.

With the cell culture system using a separator for cell configured as above, by repeating the four processes of the (1) filtering process, (2) filtrate discharge process, (3) medium transfer process, and (4) washing by back stream process as the above-mentioned separation method of the living cell, continuous culture can be carried out. In this cell culture system using a separator for cell, a rotation speed of the rotating filtration filter 10 in the washing by back stream operation is preferably set larger than a rotation speed in the filtering process. By carrying out the washing by back stream while the rotating filtration filter 10 is rotated at a high speed, the washing by back stream liquid can be spread uniformly over the entire surface of an inner face of the filter 4. Also, the washing action by a centrifugal force generated by rotation is also added, and a greater washing by back stream effect can be obtained. With the cell culture system using a separator for cell according to the present invention, continuous culture for a long time can be continued without causing clogging in the filter 4. Also, by rapid blowing-in of air into the culture vessel 31 carried out incidental to the washing by back stream process can destroy a foam layer formed on the liquid level. As a result, according to the cell culture system using a separator for cell according to the present invention, productivity of the target products can be further improved.

1. A separation system for cell comprising:
separating means for separating a cell from a culture broth containing the cell;
a buffer tank connected to the separating means through a first pipeline;
at least one or more liquid reservoir tank connected to the buffer tank through a second pipeline;
a first valve disposed on the first pipeline and controlling communication between the separating means and the buffer tank; and
a second valve disposed on the second pipeline and controlling communication between the buffer tank and the liquid reservoir tank, wherein
the first pipeline is released by the first valve so as to have the separating means and the buffer tank communicate with each other, the culture broth is aspirated to the separating means by an internal pressure difference between the buffer tank and the separating means, the first pipeline is blocked by the first valve and the second pipeline is released by the second valve so as to have the buffer tank and the liquid reservoir tank communicate with each other, and the culture broth after separation of the cell is transferred to the liquid reservoir tank by the internal pressure difference between the buffer tank and the liquid reservoir tank.

2. The separation system for cell according to claim 1, further comprising:
a liquid reservoir tank for washing by back stream connected to the buffer tank through a third pipeline; and
a third valve disposed on the third pipeline and controlling communication between the buffer tank and the liquid reservoir tank for washing by back stream, wherein
the third pipeline is released by the third valve so as to have the buffer tank and the liquid reservoir tank for washing by back stream communicate with each other, a solution for washing by back stream is supplied to the buffer tank by an internal pressure difference between the buffer tank and the liquid reservoir tank for washing by back stream, the third pipeline is blocked by the third valve and the first flow passage is released by the first valve so as to have the buffer tank and the separating means communicate with each other, the solution for washing by back stream in the buffer tank is supplied to the separating means by the internal pressure difference between the buffer tank and the separating means.

3. The separation system for cell according to claim 1, wherein
the separating means comprises a rotating filter having a filter blocking passage of a cell on an outer circumferential face of a cylindrical rotor in a container provided with a culture-broth inflow port and a culture-broth discharge port.

4. The separation system for cell according to claim 1, wherein
the separating means is disposed in a culture vessel into which a medium for culturing a cell is injected.

5. A cell culture system using a separator for cell comprising:
a separation system for cell according to claim 1; and
a culture vessel in which a culture broth of a living cell is filled, wherein
the separating means in the separation system for cell separates a filtrate not containing the cell from the culture broth containing the cell filled in the culture vessel.

6. A method of separation for cell using a separation system for cell comprising separating means for separating a cell from a culture broth containing the cell, a buffer tank connected to the separating means through a first pipeline, at least one or more liquid reservoir tank connected to the buffer tank through a second pipeline, a first valve disposed on the first pipeline and controlling communication between the separating means and the buffer tank, and a second valve disposed on the second pipeline and controlling communication between the buffer tank and the liquid reservoir tank, wherein
the first pipeline is released by the first valve so as to have the separating means and the buffer tank communicate with each other, the culture broth is aspirated to the separating means by an internal pressure difference between the buffer tank and the separating means, the first pipeline is blocked by the first valve and the second pipeline is released by the second valve so as to have the buffer tank and the liquid reservoir tank communicate with each other, and the culture broth after separation of the cell is transferred to the liquid reservoir tank by the internal pressure difference between the buffer tank and the liquid reservoir tank.

7. The method of separation for cell according to claim 6, wherein
the separation system for cell further comprises a liquid reservoir tank for washing by back stream connected to the buffer tank through a third pipeline; and
a third valve disposed on the third pipeline and controlling communication between the buffer tank and the liquid reservoir tank for washing by back stream, wherein
the third pipeline is released by the third valve so as to have the buffer tank and the liquid reservoir tank for washing by back stream communicate with each other, a solution for washing by back stream is supplied to the buffer tank by an internal pressure difference between the buffer tank and the liquid reservoir tank for washing by back stream, the third pipeline is blocked by the third valve and the first flow passage is released by the first valve so as to have the buffer tank and the separating means communicate with each other, the solution for washing by back stream in the buffer tank is supplied to the separating means by the internal pressure difference between the buffer tank and the separating means.

8. The method of separation for cell according to claim 6, wherein
the separating means in the separation system for cell comprises a rotating filter having a filter blocking passage of a cell on an outer circumferential face of a cylindrical rotor in a container provided with a culture-broth inflow port and a culture-broth discharge port.

9. The method of separation for cell according to claim 6, wherein
the separating means in the separation system for cell is disposed in the culture vessel.
